(19) United States
(12) Patent Application Publication
(10) Pub. No.: US 2010/0311083 A1
(43) Pub. Date: Dec. 9, 2010

(54) METHOD FOR DETERMINING THE THERAPEUTIC EFFECTIVENESS OF SUBSTANCES

(76) Inventors: Harald Gollnick, Magdeburg (DE); Bernd Bonnekoh, Magdeburg (DE); Raik Böckelmann, Magdeburg (DE); Lars Philipsen, Magdeburg (DE); Mandy Könnecke, Hermsdorf (DE); Ansgar Josef Pommer, Magdeburg (DE); Anja Bastian, Magdeburg (DE); Sebastian Bartsch, Berlin (DE); Yanina Schulze, Meschede (DE)

Correspondence Address: LATIMER INTELLECTUAL PROPERTY LAW, LLP, P.O. BOX 711200, HERNDON, VA, 20171, US

(21) Appl. No.: 12/282,687

(22) PCT Filed: Mar. 19, 2007
(86) PCT No.: PCT/EP2007/002418

§ 371 Date: Apr. 28, 2009

(30) Foreign Application Priority Data
Mar. 17, 2006 (DE) 10 2006 012 613.0

(51) Int. Cl. G01N 33/53

(52) U.S. Cl. 435/7.23; 435/7.2; 435/7.21

(57) ABSTRACT

The present invention relates to a method for determining the therapeutic efficacy of substances containing as agent at least one therapeutically effective, biotechnologically generated protein and/or peptide. The invention further relates to a kit and a biochip for conducting the method according to the present invention.

### CROSS-REFERENCE TO RELATED APPLICATIONS

This application is a U.S. National Stage application of PCT/EP2007/002418, filed 19 Mar. 2007, the entire disclosure of which is hereby incorporated herein by reference in its entirety.

### BACKGROUND OF THE INVENTION

1. Field of the Invention

The present invention relates to the fields of medicine and biotechnology. In particular, the present invention relates to assays for analyzing biopharmaceuticals and biological agents for their medicinal effectiveness.

2. Discussion of Related Arts

Autoimmune diseases are widely spread among the population and become manifest in the most various forms. For example, in Germany, about 1% of the population suffers from rheumatoid arthritis, which is the most common among inflammatory rheumatic diseases. Also widely spread are diseases of the skin, like for example atopic dermatitis (neurodermatitis) or psoriasis. In central Europe, about 2-3% of the population are affected by psoriasis, and in the U.S., about 4-5% are affected. Psoriasis is a chronic inflammation of the skin that is characterized by abnormal proliferation of epidermal cells (hyperplasia) and increased cutaneous blood flow. In addition to affecting the fingernails, it also leads to a joint disease, the so-called psoriatic arthritis, in a number of patients. Treatment options depend on the development of the disease in the individual patient and often require a combination of several medicaments. Besides physical therapies like UV radiation, there are preparations with different points of attack, ranging from tar or nurturing ointments to the administration of vitamin A or D derivatives (for example Zorac®, Daivonex®) and to the administration of hormones. The side effects occurring with these treatments, in particular in cases of long-term application, often limit their applicability to the individual patient and create a demand for alternative therapeutic approaches.

Various research results suggest a central involvement of T-cells in the pathophysiology of the disease. This led to the application of so-called “biologics” in the treatment of psoriasis and other autoimmune diseases. Biologics are a comparatively novel class of medicaments mainly containing biotechnologically prepared proteins having therapeutic effects. Therefore, biologics can function as antibodies, ligands, or receptors within the organ system. Their great advantage over the present forms of therapy lies in their highly specific interaction with defined molecular target structures. By means of applying biologics it becomes, on the one hand, possible to provide medicaments offering an additional possibility of treatment where all other therapeutic approaches have failed; on the other hand, the side effects often occurring with other forms of therapy can largely be avoided. Efalizumab (Raptiva®) is a biologic that is, among other things, employed for the treatment of psoriasis. This biologic is a humanized monoclonal IgG1 antibody that is directed against the molecule CD11a. Subsequently to its non-covalent association with CD18 (β2 integrin), CD11a (α-L integrin) forms the heterodimeric LFA-1 (Lymphocyte Function-associated Antigen-1), which in turn binds CD54 (ICAM-1). LFA-1 is expressed on lymphocytes, monocytes, and neutrophils and plays a decisive role in cell-to-cell adhesion processes. In clinical studies, Efalizumab led to a significant amelioration of the disease within 12 weeks in 30-40% of the patients and thus has become a novel and promising form of therapy for at least a number of patients.

Known diagnostic and predictive approaches utilize genetic methods for determining specific polymorphisms in the genome of the individual patient. In certain cases, the results thus obtained can be used for deriving the individual patient's susceptibility to the disease in question. From WO/2005/112568, a method for diagnosing or predicting a patient's response to a treatment with T cell-reducing medicaments is known. With the aid of that method, different haplotypes are thus identified, which implies that the treatment with Alefacept (Amevive®) is promising. Alefacept also belongs to the group of biologics and can be used for the treatment of psoriasis. The disadvantage of such DNA-based methods, however, is their complexity and their limitation to diseases associated with T cells. Furthermore, biologics do not have the same effects in all patients. To date, predicting the theoretical efficacy for the individual patient is hardly possible and is performed according to the principle of trial and error. Accordingly, determining the optimal dosage for individual cases and suitably monitoring a therapy ahead of treatment has up to now been impossible.

Due to the possibility that the use of biologics as immune suppressants may have severe side effects, the development of a reliable prediction method is of crucial importance in order to avoid ineffective therapeutic application.

### SUMMARY OF THE INVENTION

It is therefore a problem underlying the present invention to provide a method for determining the therapeutic efficacy of substances containing as agent at least one therapeutically effective, biotechnologically generated protein and/or peptide (biologics), where the method allows for statements on the basic efficacy of one or more therapeutically effective, biotechnologically generated protein(s) and/or peptide(s) for the individual patient ahead of treatment and/or rendering possible the monitoring of the course of therapeutic treatment. Furthermore, it is a problem underlying the present invention to provide a kit and a biochip for conducting the method according to the present invention.

These problems are solved by the methods, kits, and biochips of the present invention. More specifically, in general, the present invention relates to a method for determining the therapeutic efficacy of substances containing as an agent at least one therapeutically effective, biotechnologically generated protein and/or peptide. In particular, the present invention relates to a method for determining the therapeutic efficacy of substances containing as agent at least one therapeutically effective, biotechnologically generated protein and/or peptide, wherein the method comprises the following steps: a) providing a cell, blood, or tissue sample of human or animal origin or a sample of microbial origin, wherein the cell, blood, or tissue sample or the microbial sample is present in a predefined biological state and has target structures that are relevant for the at least one therapeutically effective, biotechnologically generated protein and/or peptide; b) labeling the at least one therapeutically effective, biotechnologically generated protein and/or peptide while maintaining its in vivo binding properties; c) applying a reagent solution containing the at least one labeled, therapeutically effective protein and/or peptide onto the sample, wherein applying the reagent solution is conducted under predefined application conditions and with predefined concentrations of the labeled, therapeutically effective and biotechnologically generated protein and/or peptide; d) determining the binding behavior of the therapeutically effective, biotechnologically generated protein and/or peptide at one or more relevant target structures of the sample; and e) evaluating the binding behavior determined in step d) with respect to the efficacy of the at least one therapeutically effective, biotechnologically generated protein and/or peptide in the examined cell, blood, or tissue sample or in the microbial sample. The present invention further relates to a kit and a biochip for conducting the method according to the present invention.

The advantage that can be achieved by means of the invention lies, among other things, in the fact that the method according to the present invention yields detailed understanding of the actual properties in vivo of effects and side effects of biologics within the organ system on the basis of binding studies in vitro and thereby allows for both predicting the efficacy of the medicament containing the one or more biologics in question for the individual patient and for the directed conduction and monitoring of a therapy in case of proven efficacy of the medicament. Due to the possibility of prediction, pharmacological/pharmaceutical development processes can, in addition, be significantly abbreviated in an advantageous manner. Furthermore, it allows the reduction of animal tests and clinical studies. Here, in an advantageous embodiment of the method according to the present invention, the application conditions according to procedural step c) comprise the variation of carrier solutions, temperature and pressure conditions. At least one of the therapeutically effective, biotechnologically generated proteins and/or peptides is selected from the group: efalizumab, alefacept, infliximab, etanercept, basiliximab, daclizumab, muromonab, trastuzumab, ibritumomab, bevacizumab, cetuximab, rituximab, omalizumab, alemtuzumab, adalimumab, or polyclonal antibodies. Polyclonal antibodies can be, for example, an anti-T cell immune serum from horse or an anti-human T cell immune serum from rabbit. The possibility of predicting and selecting effective medicaments ahead of treatment and the exact controllability of their therapeutic application furthermore advantageously leads to a significant reduction of costs in this field.

In another advantageous embodiment of the method according to the present invention, labeling is conducted according to procedural step b) by means of covalently binding one or more fluorochromes and/or covalently binding one or more biotin molecules and/or covalently binding one or more digoxigenin molecules and/or by means of labeling with a secondarily labeled antibody and/or a radioactive label to the therapeutically effective, biotechnologically generated protein and/or peptide. Fluorescein isothiocyanate (FITC), for example, is a fluorochrome that is suitable for labeling.

In another advantageous embodiment of the method according to the present invention, at least one spacer is inserted between the at least one therapeutically effective, biotechnologically generated protein and/or peptide and the at least one label in case a direct labeling of the therapeutically effective, biotechnologically generated protein and/or peptide is not possible or would lead to an interference with its binding properties in vivo.

In other advantageous embodiments of the method according to the present invention, determining the binding behavior of the therapeutically effective, biotechnologically generated protein and/or peptide at the one or more relevant target structure/s of the samples according to procedural step d) comprises detecting at least one labeling pattern of the labeled protein and/or peptide. Herein, for quantifying the efficacy of the at least one therapeutically effective, biotechnologically generated protein and/or peptide that is bound to the target structure(s), the signal strength of the labeling pattern is determined and compared to the signal strength of at least one reference sample, which has been determined under the same experimental conditions, wherein the reference sample is a cell, blood, or tissue sample or a sample of microbial origin that is comparable to the examined sample. Herein, the reference sample can be present in a biological state that is comparable to that of the examined sample or in a further biological state that differs from the state of the examined samples. Thus, the biological states comprise healthy or disease-related states of cell, blood, or tissue samples or microbial samples. By way of example, the cell or tissue sample can be a skin tissue that is affected by psoriasis, an inflammatory skin disease, a skin tumor, an inflammatory tissue or a tumor tissue and the reference sample is a normal, healthy skin tissue.

In a further advantageous embodiment of the method according to the present invention, the relevant target structures of the cell, blood, or tissue samples and/or of the reference samples are determined by an automated method by means of repeatedly applying reference solutions, each of which comprises at least one labeling molecule. After exposure to a reference solution, at least one labeling pattern is automatically detected in each case, wherein the detected labeling patterns are combined to form a complex molecular combination pattern of the cell, blood, or tissue samples or microbial samples and/or reference samples. Such a method is described in EP 0 810 428 or in DE 197 09 358.

A kit according to the present invention for conducting the methods described in the above comprises at least one therapeutically effective, biotechnologically generated protein and/or peptide and at least one labeling molecule, wherein the labeling molecule does not influence the in vivo binding properties of the protein and/or peptide. Herein, the labeling molecule can comprise fluorochrome and/or biotin molecules and/or digoxigenin molecules as well as additional secondary antibodies. Herein, the therapeutically effective, biotechnologically generated protein and/or peptide is selected from the following group: efalizumab, alefacept, infliximab, etanercept, basiliximab, daclizumab, muromonab, trastuzumab, ibritumomab, bevacizumab, cetuximab, rituximab, omalizumab, alemtuzumab, adalimumab, or polyclonal antibodies. Polyclonal antibodies can, for example, be an anti-T cell immune serum from horse or an anti-human T cell immune serum from rabbit.

Within the scope of the present invention, there is furthermore provided a biochip for conducting the methods described above, wherein the biochip comprises a cell, blood, or tissue sample of human or animal origin or a sample of microbial origin and the sample is present in a predefined biological state and has relevant target structures for at least one therapeutically effective, biotechnologically generated protein and/or peptide. Herein, the therapeutically effective, biotechnologically generated protein and/or peptide is selected from the following group: efalizumab, alefacept, infliximab, etanercept, basiliximab, daclizumab, muromonab, trastuzumab, ibritumomab, bevacizumab, cetuximab, rituximab, omalizumab, alemtuzumab, adalimumab, or polyclonal antibodies. Polyclonal antibodies can, for example, be an anti-T cell immune serum from horse or an anti-human T cell immune serum from rabbit.

Further details and areas of application can be derived from the following description of several embodiments of the method according to the present invention.

In a preferred embodiment, the therapeutically effective, biotechnologically generated protein and/or peptide is efalizumab and the cell and tissue sample is a skin sample that was fixed while conserving the relevant target structures. The predefined biological state of the skin sample is acutely psoriatic. Efalizumab is covalently labeled with fluorescein-5-ex-succinimidyl ester while maintaining its binding properties in vivo. The labeled efalizumab is dissolved, wherein the experimental conditions are selected in such a way as to correspond as far as possible to the situation in vivo within the organ system. By means of an automated method, the dissolved, labeled efalizumab as well as, optionally, at least one further dissolved labeling molecule, which is directed against various cell types and/or against structural components of the extracellular matrix and/or against structures that are characteristic for cell proliferation, activation, and adhesion, are applied onto the skin sample in succession. The fluorescence labeling pattern is detected in each cycle. The individual labeling patterns are digitalized and processed. Herein, digitalizing and quantifying the detected signals is conducted according to a method as is described, for example, in EP 1 181 525.

Firstly, a phase contrast image of a skin tissue sample is taken. Subsequently, the automatic detection of the molecular patterns for single and/or multi-epitopes is conducted. The images are rendered accessible by means of the mentioned digitalization of a computer-aided application. The phase contrast image detected prior to recording the fluorescence images is used for exact, pixel-accurate overlay of the corresponding fluorescence images. The images aligned in this manner are subjected to a correction of background and faulty exposure in a second step and the potentially present artefacts and/or faulty pixels are finally removed in a third step. This is done by means of applying a mask operation that labels faulty signals as invalid and excludes them from the subsequent evaluation.

The digitalized, processed images are binarized under the supervision of a person skilled in the art. Overlaying the binarized images for each of the detected epitopes yields a matrix of the molecular signals corresponding to binary-coded vectors that follow the Boolean logic (1=yes/true, 0=no/false) for each pixel of the digitalized visual field. In case of an area of 450×450 nm² with 20× optical zoom, one pixel represents the topographic micro unit (TMU). Under these conditions, the area of the digitalized visual field amounts to 2000×2000 pixels at most. The frequency of the occurring pixel signals for the detected combinatory labeling patterns is put in relation to the horizontal width of the skin sample. In this manner, it is possible to take into account the vertical gradient of the examined skin sample, which is normalized over tissue in a state of transition, growth, and differentiation. As suitable parameter for quantifying the expression and coexpression of epitopes, the parameter of “pixel events normalized to horizontal skin width” is obtained, referred to as PEN in the following.

The relevant target structures of the skin sample are located mainly in the dermal compartment. For determining the binding behavior of the labeled efalizumab in this compartment, the detected, digitalized and processed labeling patterns are employed. Evaluating the determined binding behavior with respect to the efficacy of efalizumab within the actual organ relation in vivo is conducted by means of comparing the identification and quantification data in vitro for the determined efalizumab binding structures to the identification and quantification data that are typical for acutely psoriatic skin samples responsive to efalizumab. Herein, the scope of quantification values that is limited with the aid of the further labeling molecules ranges between 1.1×10³ PEN for MPO and 115×10³ PEN for pan-CK and CD138. The expression of efalizumab binding structures in acutely psoriatic skin samples is up to 15 times higher as compared to non-affected skin samples from a patient suffering from acute psoriasis and up to 32 times higher as compared to healthy skin. The quantitative values are about 39.697±10.263 PEN. The presence of efalizumab binding structures with quantitatively increased PEN values as compared to healthy skin indicates the basic efficacy of efalizumab for the individual patient and additionally allows for determining an optimal, individually adjusted dosage via the quantification data.

In a further application of the invention, the therapeutically effective, biotechnologically generated protein and/or peptide is efalizumab and the cell or tissue sample is a skin sample that was fixed while conserving the relevant target structures. The predefined biological state of the skin sample is a non-affected skin sample from a patient suffering from acute psoriasis. With basic efficacy of efalizumab, a quantitative increase in efalizumab binding structures of up to 3 times as compared to healthy reference skin is found in a non-affected skin sample from an acutely psoriatic patient, exhibiting PEN values of 2.598±2.448.

In a further application of the invention, the therapeutically effective, biotechnologically generated protein and/or peptide is efalizumab and the cell or tissue sample is a skin sample that was fixed while conserving the relevant target structures. The predefined biological state of the skin sample is a healthy skin sample from a patient not suffering from psoriasis. Quantitatively determining the efalizumab binding structures in healthy skin yields values of 1.205±0.873 PEN.

In a further application of the invention, the therapeutically effective, biotechnologically generated protein and/or peptide is efalizumab and the cell or tissue sample is a skin sample that was fixed while conserving the relevant target structures. The predefined biological state of the skin sample is an acutely psoriatic skin sample. The relevant cellular target structures are located in T-lymphocyte subpopulations that are positive for CD3, CD8, CD4, CD45RA, CLA, and CD45R0. Determining the localization of efalizumab binding structures on the basis of TMU leads to colocalization values of up to 84% with CD3⁺-, up to 80% with CD8⁺-, up to 93% with CD4⁺∩CD3⁺-, up to 54% with CD45RA⁺-, up to 73% with CLA⁺-, and up to 87% with CD45R0⁺-T-lymphocytes. In this manner, the basic efficacy of efalizumab for the specific patient can be determined via the properties of specific T-lymphocytes as target structure of efalizumab during its therapeutic application.

1. A method for determining the therapeutic efficacy of substances containing as an agent at least one therapeutically effective, biotechnologically generated protein and/or peptide, said method comprising:
a) providing a cell, blood, or tissue sample of human or animal origin or a sample of microbial origin, wherein the cell, blood, or tissue sample or the microbial sample is present in a predefined biological state and has target structures that are relevant for the at least one therapeutically effective, biotechnologically generated protein and/or peptide;
b) labeling the at least one therapeutically effective, biotechnologically generated protein and/or peptide while maintaining its binding properties in vivo;
c) applying a reagent solution containing the at least one labeled, therapeutically effective protein and/or peptide onto the sample, wherein applying the reagent solution is conducted under predefined application conditions and with predefined concentrations of the labeled, therapeutically effective and biotechnologically generated protein and/or peptide;
d) determining the binding behavior of the therapeutically effective, biotechnologically generated protein and/or peptide at one or more relevant target structures of the sample; and
e) evaluating the binding behavior determined in d) with respect to the efficacy of the at least one therapeutically effective, biotechnologically generated protein and/or peptide in the examined cell, blood, or tissue sample or microbial sample.

2. The method according to claim 1, wherein at least one of the therapeutically effective, biotechnologically generated proteins and/or peptides comprises efalizumab, alefacept, infliximab, etanercept, basiliximab, daclizumab, muromonab, trastuzumab, ibritumomab, bevacizumab, cetuximab, rituximab, omalizumab, alemtuzumab, adalimumab, or polyclonal antibodies.

3. The method according to claim 1, wherein labeling according to procedural step b) is conducted by means of covalently binding one or more fluorochromes and/or covalently binding one or more biotin molecules and/or covalently binding one or more digoxigenin molecules and/or by means of labeling with a secondary labeled antibody and/or a radioactive label to the therapeutically effective, biotechnologically generated protein and/or peptide.

4. The method according to claim 3, wherein at least one fluorochrome that is used for labeling comprises fluorescein isothiocyanate (FITC).

5. The method according to claim 1, wherein at least one spacer is inserted between the at least one therapeutically effective, biotechnologically generated protein and/or peptide and the at least one label.

6. The method according to claim 1, wherein the application conditions according to procedural step c) comprise the variation of carrier solutions, temperature, and pressure conditions.

7. The method according to claim 1, wherein determining the binding behavior of the therapeutically effective, biotechnologically generated protein and/or peptide at the one or more relevant target structures of the sample according to procedural step d) comprises detecting at least one labeling pattern of the labeled protein and/or peptide.

8. The method according to claim 7, wherein the signal strength of the labeling pattern is determined for quantifying the efficacy of the at least one therapeutically effective and biotechnologically generated protein and/or peptide that is bound to the one or more target structures and that said signal strength is compared to the signal strength of a labeling pattern of at least one reference sample, which was determined under the same experimental conditions, wherein the reference sample is a cell, blood, or tissue sample or a microbial sample that is comparable to the examined sample.

9. The method according to claim 8, wherein the reference sample is present in a biological state that is comparable to that of the examined sample.

10. The method according to claim 8, wherein the reference sample is present in a further biological state that differs from that of the examined samples.

11. The method according to claim 9, wherein the biological states represent healthy or disease-related biological states of cell, blood, or tissue samples or microbial samples.

12. The method according to claim 11, wherein the cell or tissue sample is a skin tissue that is affected by psoriasis, an inflammatory skin disease, a skin tumor, an inflammatory tissue or a tumor tissue and the reference sample is a normal, healthy skin tissue.

13. The method according to claim 1, wherein the relevant target structures of the cell, blood, or tissue samples or of samples of microbial origin and/or of the reference samples are determined by an automated method by means of repeatedly applying reference solutions, each of which comprises at least one labeling molecule and at least one labeling pattern is automatically detected in each case after exposure to a reference solution, wherein the detected labeling patterns are combined to form a complex molecular combination pattern of the cell, blood, or tissue samples and/or reference samples.

14. A kit for conducting the method according to claim 1, said kit comprising at least one therapeutically effective, biotechnologically generated protein and/or peptide and at least one labeling molecule, wherein the labeling molecule does not influence the binding properties in vivo of the protein and/or peptide.

15. The kit according to claim 14, wherein the labeling molecule comprises fluorochromes and/or biotin molecules and/or digoxigenin molecules as well as secondary antibodies.

16. The kit according to claim 14, wherein the at least one protein and/or peptide comprises efalizumab, alefacept, infliximab, etanercept, basiliximab, daclizumab, muromonab, trastuzumab, ibritumomab, bevacizumab, cetuximab, rituximab, omalizumab, alemtuzumab, adalimumab, or polyclonal antibodies.

17. A biochip for conducting the method according to claim 1, said biochip comprising a cell, blood, or tissue sample of human or animal origin or a sample of microbial origin, wherein the sample is present in a predefined biological state and has relevant target structures for at least one therapeutically effective, biotechnologically generated protein and/or peptide.

18. The biochip according to claim 17, wherein the at least one protein and/or peptide comprises efalizumab, alefacept, infliximab, etanercept, basiliximab, daclizumab, muromonab, trastuzumab, ibritumomab, bevacizumab, cetuximab, rituximab, omalizumab, alemtuzumab, adalimumab, or polyclonal antibodies.
